(19) United States
(12) Patent Application Publication
(10) Pub. No.: US 2026/0348896 A1
(43) Pub. Date: Nov. 5, 2026

(54) SENSOR INCLUDING MEASUREMENT CIRCUITS FOR DETERMINING RESISTANCE AND CAPACITANCE OF AN ENVIRONMENTAL CONDITION AND A METHOD OF OPERATING SUCH A SENSOR

(71) Applicant: P.E.S. Technologies Limited, Diss (GB)

(72) Inventors: Jim BAILEY, Diss (GB); Graham BAILEY, Diss (GB); Matthew JACKSON, Ely (GB); Keith BROOKES, Cambridge (GB); Andrew HAYES, Cambridge (GB)

(21) Appl. No.: 19/118,745

(22) PCT Filed: Oct. 3, 2023
(86) PCT No.: PCT/GB2023/052543

§ 371 Date: Apr. 4, 2025

(30) Foreign Application Priority Data
Oct. 4, 2022 (GB) 2214575.9

(51) Int. Cl. G01N 27/04; G01N 27/22; G01N 33/24

(52) U.S. Cl. CPC G01N 27/045; G01N 27/043; G01N 27/228; G01N 33/24

(57) ABSTRACT

A sensor is provided for measuring an environmental condition. The sensor includes a sensing element having a resistance and a capacitance which are sensitive to the environmental condition; a first measurement circuit for determining the resistance of the sensing element using logarithmic scaling; a second measurement circuit including an integrator amplifier for determining the capacitance of the sensing element; and a switching circuit for switching the sensing element in turn into the first and second measurement circuits such that the resistance and capacitance can be determined within a predetermined time period.

### FIELD

The present application relates to an electronic sensor for measuring an environmental condition and a method of operating such a sensor.

### BACKGROUND

Electronic sensors are widely used for determining an environmental condition. As a simple example, temperature may be measured electronically by passing current from a constant current supply through a conductive wire having a resistance which is dependent on temperature. The resistance of the wire can then be determined by measuring the voltage across the wire, which in turn provides a measurement of temperature.

In this simple example, the environmental condition to be measured comprises a single physical parameter (temperature) which allows for a relatively straightforward implementation of the electronic sensor. However, in other cases, the environmental condition to be measured may be more complex, comprising multiple physical parameters, such as the respective concentrations of different gases (volatiles) produced in a given situation, for example, exhaust products from the combustion of fuel or decay emissions resulting from a biological process.

Sensors for detecting the odours in a mixture of various gases are sometimes referred to as an electronic nose. Such devices are discussed in “White Paper-How Does An Electronic Nose Work” by Lalberte and Porter, January 2017, available from https://www.researchgate.net/publication/320455411, which provides information on various types of sensor for detecting odours.

In order to provide a better determination of such a complex environmental condition, the sensing operation may include measuring both resistance and capacitance of a sensor (or a particular component thereof, typically referred to as a transducer or sensing element). Furthermore, such measurements may be made repeatedly over a given time period to provide an indication of the temporal evolution of the environmental condition.

However, existing sensors for making such measurements of a complex environmental condition are generally complex (and hence expensive) devices, or else are simpler devices that are limited in terms of performance, such as providing limited measurement accuracy and/or poor time resolution.

### SUMMARY

The invention is defined in the appended claims.

A sensor is provided for measuring an environmental condition. The sensor includes a sensing element having a resistance and a capacitance which are sensitive to the environmental condition; a first measurement circuit for determining the resistance of the sensing element using logarithmic scaling; a second measurement circuit including an integrator amplifier for determining the capacitance of the sensing element; and a switching circuit for switching the sensing element in turn into the first and second measurement circuits such that the resistance and capacitance can be determined within a predetermined time period.

In some implementations, the first measurement circuit comprises a log amplifier.

In some implementations, the first measurement circuit is configured to measure resistance over at least six decades, preferably over at least seven decades, preferably over eight decades.

In some implementations, the first measurement circuit is configured to measure resistance over a range having a lower limit of no greater than 2500 ohms, preferably no greater than 1000 ohms, and preferably no greater than 500 ohms, and an upper limit of no less than 1 gOhm, preferably no less than 5 gOhm, and preferably no less than 25 gOhm.

In some implementations, the integrator amplifier comprises an op amp integrator.

In some implementations, the sensor is configured to provide a step change voltage to the integrator amplifier, and the slope of the output from the integrator amplifier is dependent on the capacitance to be measured.

In some implementations, the second measurement circuit further comprises a time to digital converter (TDC), and optionally wherein the TDC may be used to measure the slope of the output from the integrator amplifier.

In some implementations, the sensor is configured to apply a correction to the measured capacitance based on the measured resistance.

In some implementations, the correction is only applied if the measured resistance is below a threshold value, optionally wherein the threshold value is below 100 kOhms, for example, below 40 kOhms.

In some implementations, the second measurement circuit is configured to measure capacitance over a range having a lower limit of no greater than 5 F, preferably no greater than 1 F, and an upper limit of no less than 100 F, preferably no less than 200 F.

In some implementations, the operation of the first measurement circuit for determining resistance is performed independently from, and sequentially with, the operation of the second measurement circuit for determining capacitance.

In some implementations, the resistance and capacitance can be determined within a predetermined time period of 0.1 s, for example within 0.01 s, for example within 1 ms.

In some implementations, the sensor further comprises at least one channel for providing a known resistor for measurement by the first measurement circuit and/or for providing a known capacitor for measurement by the second measurement circuit as a mode of calibration.

In some implementations, the sensor comprises multiple sensing elements, optionally wherein the number of sensing elements is in a range from 1 up to 6, optionally within a range from 1 up to 20, or optionally with a range from 1 up to 50.

In some implementations, the sensor is configured to switch each sensing element in turn into the first and second measurement circuits.

In some implementations, the sensing elements are repeatedly switched in turn into the first and second measurement circuits, and the time duration for all the sensing elements to be switched in turn into the first and second measurement circuits for measuring the resistance and capacitance of all the sensing elements is no greater than 1 second, for example no greater than 0.1 second, for example no greater than 20 ms.

In some implementations, the sensing element comprises an electronic nose comprising a polymer semiconductor-based sensor array.

In some implementations, the environmental condition to be measured corresponds to soil health.

The present disclosure further provides a method of using a sensor to measure an environmental condition, the sensor including a sensing element having a resistance and a capacitance which are sensitive to the environmental condition. The method comprises: switching the sensing element into a first measurement circuit; determining with the first measurement circuit the resistance of the sensing element using logarithmic scaling; switching the sensing element into a second measurement circuit; and determining with the second measurement circuit the capacitance of the sensing element using an integrator amplifier.

The above method may be implemented, for example, using suitable implementations of a sensor such as described above.

### BRIEF DESCRIPTION OF THE FIGURES

Various implementations of the claimed invention will now be described by way of example only with reference to the following drawings.

FIG. 1 is a schematic diagram of a sensor in accordance with the present disclosure which may be used to measure (detect) a condition of an environment.

FIG. 2 is a schematic diagram of a log amplifier which may be used as an example of a resistance detector such as shown in the sensor of FIG. 1.

FIG. 3 is a more detailed diagram (compared with FIG. 2) of a log amplifier which may be used as an example of a resistance detector such as shown in FIG. 1.

FIG. 4 is a graphical plot showing an example of the relationship between resistance of a sensing element and output from a resistance detector in a sensor such as shown in FIG. 1.

FIG. 5 is a schematic diagram of an example of a capacitance detector which may be used in the sensor of FIG. 1.

FIG. 6 is a graphical plot showing an example of the deviation of an op amp integrator from the behaviour of an ideal integrator.

FIG. 7 is a flowchart showing an example of a method for operating a sensor such as shown in FIG. 1.

### DETAILED DESCRIPTION

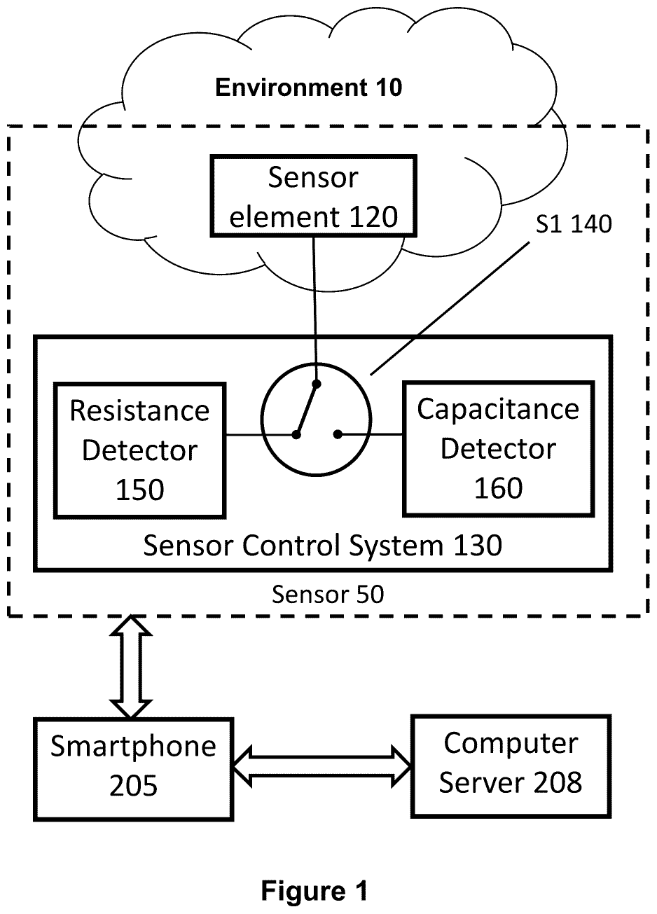

FIG. 1 is a schematic diagram of a sensor 50 which is used to measure (detect) the condition of an environment 10. The sensor 50 includes at least one sensing element 120 which acts as a transducer whereby the electrical properties of the sensing element 120 are dependent on the condition of the environment 10, such that a measurement of the electrical properties of the sensing element provides information about the condition of the environment 10.

The sensor 50 further includes a sensor control system 130 having a detector 150 for measuring the resistance of the sensing element 120 (sometimes referred to as a sensor element) and a detector 160 for measuring the capacitance of the sensing element 120. Note that in FIG. 1 the sensor control system 130 is shown as being separate from the sensing element 120, however, in many implementations the sensor control system 130 and the sensing element 120 may be combined or integrated into a single unit. Although some sensors are based on the measurement of just resistance (without any measurement of capacitance), the sensor of FIG. 1 supports the measurement of both resistance and capacitance for the sensing element(s) 120. The provision of both resistance and capacitance measurements provides better data for analysing a sample which is being sensed (especially when the analysis is performed using an artificial intelligence/machine learning system as described further below). However, the measurement of both resistance and capacitance places additional requirements on the detectors 150, 160 and the sensor control system 130, as discussed in more detail below.

The sensor control system 130 provides interface electronics for performing, storing and communicating the resistance and capacitance measurements obtained from the detectors 150, 160. As mentioned above, the sensor 50 including the sensing element(s) 120, the detectors 150, 160, and the sensor control device may be integrated into a single device. Typically such a device may be portable and able to run off a battery for an extended period of time. This in turn puts some practical limitations on the level of power consumption that can be supported for the sensor control system 130 and the sensor in general.

By way of example to provide an overview of a potential application of sensor 50, and without limitation, the sensor 50 may be used to check the properties (environmental condition) of soil, such as for use by farmers. In particular, the sensor 50 may be used to provide information on a variety of biological, chemical and physical soil health indicators, including microbial biomass and soil organic matter content, which are dependent on the activities and diversity of a soil's microbial community. It is noted that declining soil health may be lowering or limiting crop yields, decreasing the resilience of crops to pests and diseases, and making crops less resilient to drought. To support such monitoring, the sensor 50 is generally portable and capable of being held in one hand. As mentioned above, the sensor 50 may be designed to have sufficiently low power consumption to allow the sensor to run off a battery for a significant period of time, for example, an entire day.

In a typical implementation, the sensor 50 is a hand-held, battery powered device which may be controlled (for example) using an app on a smartphone 205. The sensor is used to measure and analyse soil samples directly in-field without requiring additional sample preparation. Furthermore, rather than doing multiple different tests to get information about different aspects of soil health, by using the sensor 50 to analyse the activities and diversity of the microbial community in a soil sample, information can be derived about many different soil health indicators. The measurement process and analysis may be completed within a few minutes, in effect providing real-time information about the environmental condition for the current location.

The sensor 50 may further include a global positioning system (GPS) detector and/or support any other such location monitoring system (not shown in FIG. 1). This allows each sample analysed by the sensor, and the associated measurement results measurements, to be tagged with the GPS location (and time) coordinates. A user is then able to track the variation in environmental condition (such as soil properties) over time for a particular location that is being monitored.

In operation, a small sample of soil (for example, about 100 g) may be scooped from the ground and entered into a cavity or drawer of the sensor 50 (the drawer or cavity for holding the sample is not shown in FIG. 1). The sensing element 120 may comprise a test strip which may be removed from protective packaging and inserted into the sensor 50 (either before or after the soil is entered into the sensor device 50). In some implementations, the sensing element 120 may comprise a single use test strip, whereby a new test strip is entered into the sensor 50 for each new measurement. In some implementations, the sensing element 120 may be reusable, for example with the sensor being cleaned between successive readings. In some cases the sensor cleaning may be performed in situ (within the sensor 50 itself); in other cases the sensing element 120 may be removed from the sensor 50 for cleaning prior to reuse. It will be appreciated that some sensors may support both single use and also reusable sensing elements 120.

Once the relevant measurements have been performed, in particular, when the sensor 50 has obtained a sequence of measurements of the resistance and capacitance of the sensing element(s) 120 for determining the condition of environment 10, the data may, in some implementations, be transferred to an app on a smartphone 205. This data communication between the sensor 50 and the smartphone may be performed, for example, by using a wireless Bluetooth connection. The smartphone may perform a local analysis of the measurement data and/or may send the measurement data to a remote location, such as computer server 208, to perform an analysis of the measurement data. In some implementations, the computer server may be part of (or provided by) a cloud computing service. In other implementations, the sensor 50 may be able to communicate directly with the computer server 208 over an appropriate network (not shown in FIG. 1) without going through the smartphone 205.

In some implementations, the analysis of the soil measurements may be performed on any suitable device, such as smartphone 205 or computer server 208, using an artificial intelligence (AI) or machine learning (ML) system (not shown in FIG. 1) which may be implemented on any suitable AI platform, such as TensorFlow (see https://www.tensorflow.org/) or Azure from Microsoft Corporation. The AI/ML software analyses the measurement data from the sensor 60 and may identify values for a wide range of soil health indicators. These results can then be returned from the cloud (such as computer server 208) to the smartphone 205 where they may be presented to the user for review and any appropriate action.

The sensor 50 of FIG. 1 is designed to handle measurement of high impedance sensing elements 120 such as may be used (inter alia) in electronic noses. Such sensing elements 120 may comprise a substrate, for example made from a semiconductive material such as metal oxide silicon (MOS) or may be provided as part of a polymer semiconductor-based sensor array. Further details about various types of sensing element 120 can be found for example in the White Paper cited above by Lalberte and Porter.

Although the sensor 50 shown in FIG. 1 has one sensing element, in many implementations of sensor 50 there are multiple sensing elements. In some cases, the sensing elements all have generally the same substrate, but different sensing elements may have different coatings from one another. These different coatings may then impact how particular gases interact with the substrate, and hence how they change the electrical properties (specifically resistance and capacitance) of the sensing elements 120.

In some implementations of a sensor 50 such as shown in FIG. 1, the sensor 50 has six sensing elements 120, but other sensors may have a different number of sensing elements. It will be appreciated that increasing the number of sensing elements may give greater discrimination and accuracy when measuring the environmental condition, however, increasing the number of sensing elements may also lead to a larger sensor 50 with increased power consumption. Accordingly, the number of sensing elements provided for a given sensor 50 may be selected according to the circumstances of any particular application.

If the sensor has N sensing elements, each sensing element having a respective different coating, then each sensing operation in effect results in a 2N-dimensional vector comprising values r1, c1, r2, c2 . . . rN, cN. Thus for each sensing element (1, 2, . . . N), two sensing values are obtained, namely the measured resistance of the sensing element 120 (e.g. r1) and the measured capacitance of the sensing element 120 (e.g. c1). The sensing operation therefore provides a 2N-dimensional space, and the particular location of the sensor results within the 2N-dimensional space corresponds to the determined environmental condition. (The environmental condition may be based on a multiplicity of different physical parameters, such as the respective concentrations of a number of different gases (the target analytes) generated from a sample such as a soil sample).

In many cases, a sensing operation may comprise a set (time sequence) of individual sensing measurements spread out over a period of time, for example over a few minutes. This time sequence of measurements (readings) provides additional information for measuring an environmental condition(compared with a measurement at just a single point of time). However, during the period of this time sequence, the sensing element(s) 120 may typically change in resistance, R, by orders of magnitude when the sensing element(s) is (are) exposed to the target analytes such as gases or volatile organic compounds released by the sample. In particular, the resistance tends to reduce as the gases, volatiles etc bond or adhere to the surface of the sensing element and help to provide a conductive (lower resistance) path across the sensing element. This reduction in resistance (impedance) over a few minutes may comprise (for example) a fall from a resistance of the order of gigaOhms to a resistance of the order of Ohms. Accordingly, the resistance detector 150 is designed to accommodate measurements of resistance over a wide range, as discussed in more detail below.

The capacitance to be measured by the capacitance detector 160 with respect to the sensing element(s) is typically of the order of pico-Farads. Compared with the large change of resistance across the overall measurement period of a few minutes (for example), there is typically less variation in the level of capacitance for the sensing element(s). In some implementations, although there may be some variation in the capacitance level across the overall measurement period, the capacitance of sensing element(s) may remain of the order of pico-Farads during this measurement period.

It will be appreciated that the above values of the resistance and capacitance of the sensing element(s), the length of the measurement period, and the change in the values of the resistance and capacitance of the sensing element(s) during this measurement period are provided by way of example only. Sensing operations using different sensor implementations and/or performed on other types of sample (rather than soil) may involve different values, and the sensor 50 may be designed as appropriate to accommodate such different values.

The sensor 50, including the resistance detector 150, the capacitance detector 160 and the sensor control system 130, are typically designed to support having multiple sensing elements 120. Furthermore, the various sensing elements 120 must usually be read at short time intervals because the signals (i.e. the resistance and capacitance values of the various sensing elements) may change rapidly to reflect concentration changes of the target analytes in the environment being measured. For example, the desired time sampling rate for each sensing element 120 might be of the order of milliseconds or less. Having multiple sensing elements which are read repeatedly at short intervals generally adds to the size and draw on a battery used to power the sensor 50, which can be challenging for a hand-held sensor implementation.

There are various existing approaches for measuring both resistance and capacitance for a sensing element—for example, impedance spectroscopy techniques, frequency conversion methods, and RC curve methods. Impedance spectroscopy generally involves the availability of good mathematical models, and frequency sweeps from very low to very high frequencies are generally required to achieve the best results. The resulting sensor typically comprises complex hardware that is relatively expensive.

To try and simplify the hardware and reduce costs, some implementations may focus on a few key frequencies to overcome, but there is still a need for mathematical modelling. Such modelling may be difficult where there is a lack of a physical background for a proper interpretation of the results. For example, it would be difficult to develop a suitable model for soil sampling because the interactions between the target analytes and/or non-target analytes and the sensing element(s) 120 have not been properly characterised (and such characterization would be a difficult and time-consuming task).

Measuring resistance (R) and capacitance (C) from the RC curve of a sensor is another known approach for obtaining the outputs from a sensing element, as the RC curve naturally contains information about both R and C. One limiting factor here is the time taken for the RC curve to rise to a suitable level from which the measurement data can be extracted. This is generally not an issue at low R, but does become problematic for high R. For example, plausible range limits for the resistance and capacitance of a sensing element 120 are 450 Ohms up to 25 gOhms for the resistance and 1 pFarad up to 100 pFarads for the capacitance. The combination of a capacitance of 1 pF and a resistance of 450 Ohms implies an RC value of 450 ps, which is very fast and hence difficult to measure accurately, while the combination of a capacitance of 100 pF and a resistance of 25 gOhms implies an RC value of 2.5 seconds, which for many applications is too long, because there is a lack of sufficient temporal resolution to track significant variations in the time domain.

The frequency conversion method is described, inter alia, in: “A CMOS integrated low-voltage low-power time-controlled interface for chemical resistive sensors” by Marcellis et al, November 2012, available from: https://www.researchgate.net/publication/236346603_A_CMOS_integrated_low-voltage_low-power_time-controlled_interface_for_chemical_resistive_sensors. This approach allows for a faster measurement at high R, in effect by limiting the maximum length of time a measurement can take. However, even with this approach, the measurement time at high R may be up to seconds per datapoint per sensing element, so that a complete sweep of the system may take tens of seconds. Further, the equations used in this approach are only valid in a specific region of parameter space which is narrow compared with the full range of resistance values experienced by the sensing element. The usable range can be expanded by not using the equations and instead measuring purely frequency characteristics and relating those via machine learning to the sensor response to known analyte concentrations or known properties of the analysed sample. However, this approach has a significant draw-back in that a change to the hardware electronics of the sensor 50 may require an entirely new machine learning training dataset to be constructed, because the electronic properties measured no longer correspond directly to the physical attributes of the sensor.

Rather than adopting such an existing method for measuring resistance and/or capacitance of a sensing element, such as a method based on impedance spectroscopy, RC curves or frequency conversion methods, the sensor 50 described herein adopts a different approach for performing measurements of resistance and capacitance from detectors 150, 160 respectively. In this approach the resistance and capacitance are measured separately from one another (rather than using a single measurement to extract both values). Using two separate, but efficient, measurement techniques for the resistance detector 150 and the capacitance detector 160 has been found to support quicker and more accurate results (compared with utilising a single measurement technique that tries to determine both the resistance and the capacitance of the sensing element in a single operation).

In the implementation of FIG. 1, the resistance and capacitance measurements may be performed in sequence, one at a time, using a switching system such as represented schematically by switch S1 140. For example, when a resistance measurement is to be made, the switch S1 is set to the configuration shown in FIG. 1, in which the sensing element 120 is connected to the resistance detector 150 (but not to the capacitance detector). This allows the resistance detector 150 to perform the desired measurement of the resistance of the sensing element 120.

Once this first sensing measurement of the resistance of the sensing element 120 has been completed, the setting of the switch S1 is changed to link the sensing element 120 to the capacitance detector 160 (in place of to the resistance detector). This allows the capacitance detector 160 to perform the desired measurement of the capacitance of the sensing element 120.

The operation of the switch S1 140 may be managed by the sensor control system 130. For example, the sensor control system may initially set the switch to the position shown in FIG. 1 to allow the resistance detector to perform a measurement of resistance. The resistance detector 150 may then notify the sensor control system 130 when the resistance detector 150 has completed this measurement of resistance, and the sensor control system 130 may now change the setting of switch S1 to allow a measurement of capacitance to be performed by the capacitance detector 160. It will be appreciated that this switching back and forth between the resistance detector 150 and the capacitance detector may continue over the course of the measurement period (typically a few minutes).

As mentioned above, in some implementations there may be multiple sensing elements. In this situation, a more complex switching configuration and sequence may be provided to allow the resistance detector 150 and the capacitance detector to measure each sensing element in turn. For example, if there are two sensing elements denoted SE1 and SE2, the sensor control system may control the switches 140 to measure in turn the resistance and capacitance of sensor SE1 and then to measure in turn the resistance and capacitance of SE2. Other patterns for ordering the sensing may be adopted. For example, in some implementations, the sensor control system 130 may control the switch settings so that the resistance detector 150 first determines the resistance (in turn) of each of the sensing elements 120, and the capacitance detector 160 may then determine the capacitance (in turn) of each of the sensing elements 120. Other patterns of measurement of the different sensing elements 120 may be managed by the sensor control system based on various factors such as the overall number of sensing elements and the particular circumstances of any given implementation (such as the timescale of switching by the sensor control system 130 compared against the timescale of variation in the concentrations of target analytes).

Considering in more detail the resistance detector 150, this may be configured to provide a logarithmically scaled output such as according to the following formula:

Vₒᵤₜ=k log (Vᵢₙ/V_{ref})+C (Eq 1)

in which V_{ref} is a reference voltage to which the input voltage (Vᵢₙ) is compared and k and c are constants, with k determining the gradient and c the base-line of the mapping to Vₒᵤₜ. Note that k can also be seen as a factor to alter the base of the log (for example, to shift between base 10 and natural logarithms), while c can be regarded as adjusting the value of V_{ref}. The parameters V_{ref}, k and c can be configured by the implementation to provide a suitable range of output voltages for a specific range of input voltages. For example, if the desired range of output voltage is 0-5 volts for a specified range of input voltages, then the value of k may be chosen in effect to give a 5 volt range between the lowest and highest input voltage values (for Vᵢₙ), and the value of c (and/or V_{ref}) may be chosen so that the lowest input voltage corresponds to one end of this voltage range, usually 0 volts.

To provide this logarithmic scaling, an implementation of the resistance detector may comprise a logarithmic amplifier (log amplifier), see for example:
https://en.wikipedia.org/wiki/Log_amplifier and
https://www.tutorialspoint.com/linear_integrated_circuits_applications/linear_integrated_circuits_applic ations_log_and_anti_log_amplifiers.htm.

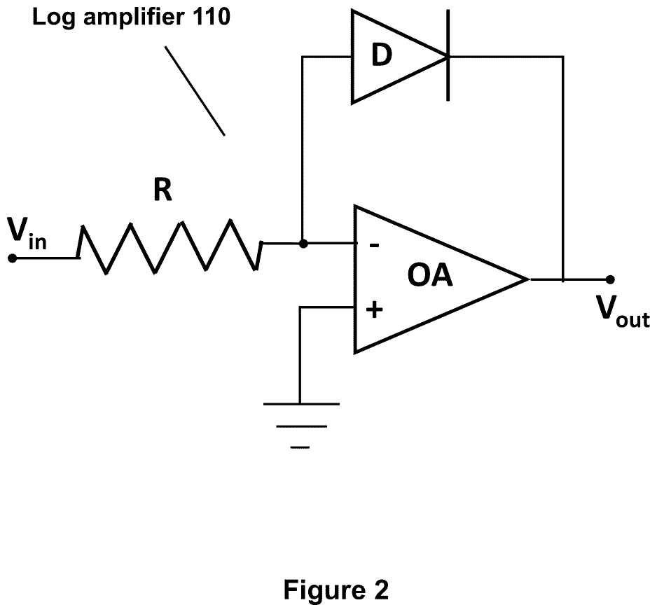

FIG. 2 is a schematic diagram of a log amplifier (log amp) 110 which may be used as an example of a resistance detector such as shown in the sensor of FIG. 1. The log amp comprises a resistance R, a diode D and an operational amplifier (op amp) OA. The relationship between the input voltage (Vᵢₙ) and the output voltage Vₒᵤₜ in FIG. 2 generally follows Equation 1 above, with the values of V_{ref}, k and c being determined, inter alia, by the value of the resistor R and the properties of the diode D.

In broad terms the grounding of the op-amp implies that the voltage is zero at both the positive and negative (inverting) inputs into the op amp, hence there is no current flow through the op amp (for an ideal device). The current I through the resistor R therefore matches the current through the diode D. The current through the resistor is given by I=Vᵢₙ/R and so scales linearly with the voltage Vᵢₙ. The voltage across the diode is (minus) Vₒᵤₜ and the current through the diode scales exponentially with this voltage, I~(exp Vₒᵤₜ) for the unsaturated region. Accordingly, the only way for the current through resistor R to match (scale linearly with Vᵢₙ) is for Vₒᵤₜ to scale logarithmically with Vᵢₙ (as per Equation 1 above) because we then have the current through the diode as I~exp Vₒᵤₜ=>I~exp (log Vᵢₙ)=>I~Vᵢₙ, so that the current through the resistor R and the current through the diode D both scale linearly with Vᵢₙ.

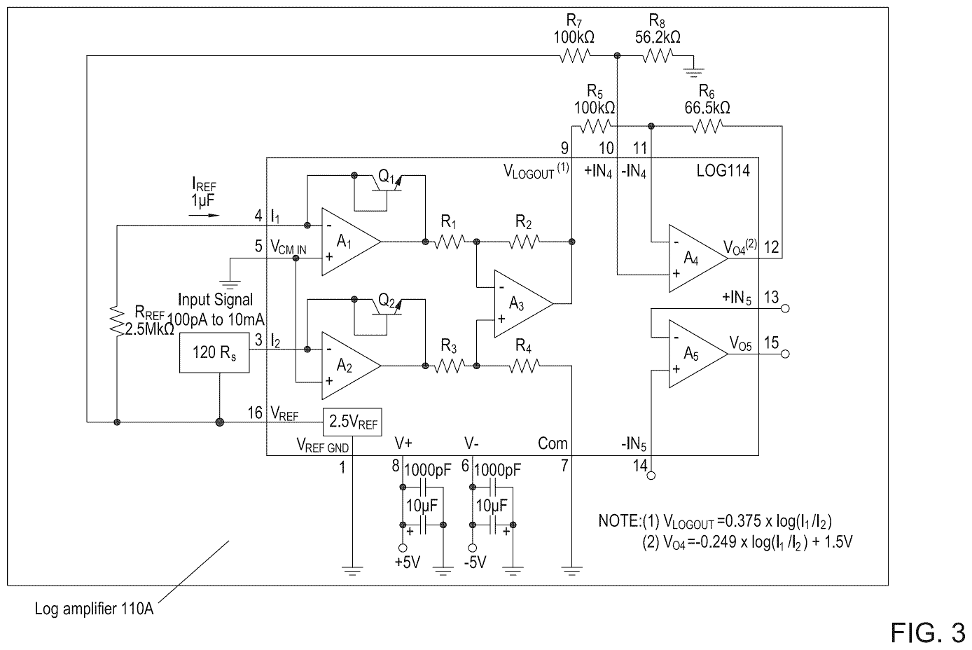

FIG. 3 illustrates a log amp 110A which again may be used in the resistance detector 150 of FIG. 1. Whereas FIG. 2 showed only core components of a logarithmic amplifier 110 to allow a high-level understanding of such a device, the log amp 110A of FIG. 3 represents a production model, namely a LOG114 logarithmic amplifier commercially available from Texas Instruments (TI), see https://www.ti.com/product/LOG114. Note that the discussion of the LOG114 device herein will be limited to those aspects of direct relevance to the sensor of the present application. Further information relating to the LOG114 device can be found in the supplier data sheet, which can be obtained from:
https://www.ti.com/lit/ds/symlink/log114.pdf? ts=1664175290746&ref_url=https%253A%252F%252Fw ww.ti.com%252Fproduct%252FLOG114 (hereinafter referred to as the Data Sheet).

FIG. 3 of the present application is based on FIG. 1 of the Data Sheet for a Dual Supply Configuration of the LO114 device which may be used to provide an output voltage that represents a logarithmic comparison of two currents. As shown in FIG. 3, the LOG114 device may be configured to have one input that provides a reference current I_{ref} (also marked as I₁) which is generated by applying a known reference voltage V_{ref} across a known reference resistance R_{ref}. This reference current I_{ref} provides the input to op amp A1 on its inverting terminal. Similarly, a second current I₂ is generated by applying the known reference voltage V_{ref} across the (unknown, to be measured) resistance Rₛ of the sensing element 120. This second current I₂ provides the input to op amp A2's inverting terminal. The positive (non-inverting) inputs of op amps A1 and A2 are both connected to ground.

Note that the logarithmic amplifiers A1, A2 use a diode connected transistor in their feedback paths, where the voltage across the diode is proportional to the logarithm of the current though it. This configuration is slightly different in design from the example of FIG. 2, because of using a transistor in the feedback loop (rather than a diode), however, the overall mode of operation remains similar to the discussion above relating to FIG. 2. Further information about the operation of a logarithmic amplifier including a transistor can be found at: https://electricalvoice.com/log-amplifier-circuit-applications/

The two voltage outputs from log amps A1 and A2 are fed to respective ports of a differential amplifier A3 which outputs a voltage representing the difference between the two. In particular, the output from log amp A1 is passed to the inverting input of A3 and the output from the log amp A2 is passed to the positive (non-inverting) input of A3. Amplifiers A4 and A5 of LOG114 are used for scaling and other various ancillary functions.

The LOG114 device operates as follows in conjunction with the present application. The reference current input I_{ref} is given by V_{ref}/R_{ref}, While the other current input 12 is given by V_{ref}/Rₛ, where Rₛ is the resistance of the sensing element 120, which is the parameter to be measured. The voltage output from log amp A1 can then be represented as V(A1)=k log V_{ref}/R_{ref}+c while the voltage output from log amp A2 can be represented as V(A2)=k log V_{ref}/Rₛ+c. (As for Equation 1 above, k and c are constants corresponding to a scaling and base-point and can be known or measured properties of the device).

The voltage output from log amp A3 is proportional to V(A2)−V(A1) (since the output from op amp A1 goes to the inverting input on A3), which in turn can be rewritten as k(log V_{ref}/Rₛ−log V_{ref}/R_{ref}). This can be simplified in turn to −k(log Rₛ/R_{ref}). This expression gives the ratio of the sensing element resistance to the reference resistance. Furthermore, since the reference resistance is known, this ratio allows the resistance of the sensing element 120 to be determined.

The overall output of the LOG114 device is generally given by:

Vₒᵤₜ=0.375 log₁₀(I₁/I₂)+V_{offset} (Eq 2)

(see Equation 2 of the Data Sheet). It will be appreciated that this has the same general format as Equation 1 above. In this configuration, the output voltage changes by 0.375 volts for each decade (power of 10) in the input current with respect to the reference current.

The use of such a log amplifier 110 as part of a resistance detector for measuring the resistance of sensing element 120 has certain benefits. Thus logarithmic amplifier 110 is able to support input (voltage or current) measurement across many decades, which in turn can lead to a measurement of resistance across many decades. This range of resistance measurements is very helpful in various application areas, such as the investigation of soil samples as discussed above. In addition, the logarithmic amplifier 110 is able to achieve a quick measurement of the resistance of a sensing element, with a timing (duration) typically in the range of μs to ms. Importantly, this timing does not depend on the RC values, but rather is generally independent of the values of R and C (resistance and capacitance). This then allows the timing performance of the resistance detector 150 to be specified as a predetermined period, for example less than 0.1 seconds, optionally less than 0.01 seconds, or optionally less than 0.001 microseconds. Since this predetermined period generally applies irrespective of resistance level, this approach provides measurement results which are more consistent and predictable.

Furthermore, even if a sensor 50 has multiple sensing elements 20, the resistance detector 150 is able to read (sequentially) measurements for each of the multiple sensing elements in a total time which is significantly under 1 second. This in turn supports a high (significantly greater than 1 Hz) frequency of measurements for each of the multiple sensing elements.

As noted above, the LOG114 logarithmic amplifier 110A calculates the log ratio of an input current to a reference current for the dual supply configuration of FIG. 1 of the Data Sheet. (The LOG114 logarithmic amplifier 110A also supports a single supply configuration). The LOG114 device has two log amplifier sections A1, A2 to compensate for various factors such as temperature using a differential amplifier A3. There are additional op amps within the LO114 device to provide scaling, offsetting, filtering, and so on.

The resistance of the sensing element 120 is typically in the range from 500 Ohms up to 25 gOhms (although this varies according to the type of sample that is being investigated). Since current is inversely proportional to resistance (for a fixed voltage), the logarithmic range of the current input accepted by the logarithmic amplifier 110 should correspond approximately to the logarithmic range of the resistances to be measured in order to provide measurements across the range of interest for resistance. This condition is satisfied by the LOG114 log amplifier 110A, which supports a current input spanning some eight decades from 100 pA to 10 mA.

It is noted that if the resistance is above about 1 gOhm, there is increased susceptibility to noise which can make it harder to achieve a stable and accurate measurement (reflecting the lower level of current signals for high resistance). The careful provision of shielding and solid grounding may be used to reduce the noise level experienced within the resistance detector 150, thereby allowing accurate results to be obtained even for measurements of high resistance.

Although the above description focuses on the use of a logarithmic amplifier to provide logarithmic scaling, such logarithmic scaling can also be obtained via other forms of circuitry, such as by using a set of ‘range-resistors’. In this latter approach, different resistors are switched into the circuit using a chip that can be electronically told which way to route the circuit, so that the output of an op-amp measurement circuit is kept within a specific, measurable voltage range. The chip may comprise a multiplexer chip, or in some cases two or more such multiplexer chips. For example, a multiplexer chip in one implementation was limited to 6 channels, whereas there were 8 range resistors to switch in, namely: 10 GOhm, 1 GOhm, 100 MOhm, 10 MOhm, 1 MOhm, 100 kOhm, 10 kOhm, and 1 kOhm, so that this required two 6 channel multiplexer chips to handle the full set of range resistors. In such an implementation, the one or more multiplexer chips are used to switch the range resistor down or up based on the output voltage hitting a threshold voltage. In particular, if an output voltage is starting to go out of range, the next time the circuit is measured, a lower (or higher) range resistor is switched in as appropriate. The final output from such a circuit is therefore broadly analogous to that provided by a log amp chip 110 (such as described above).

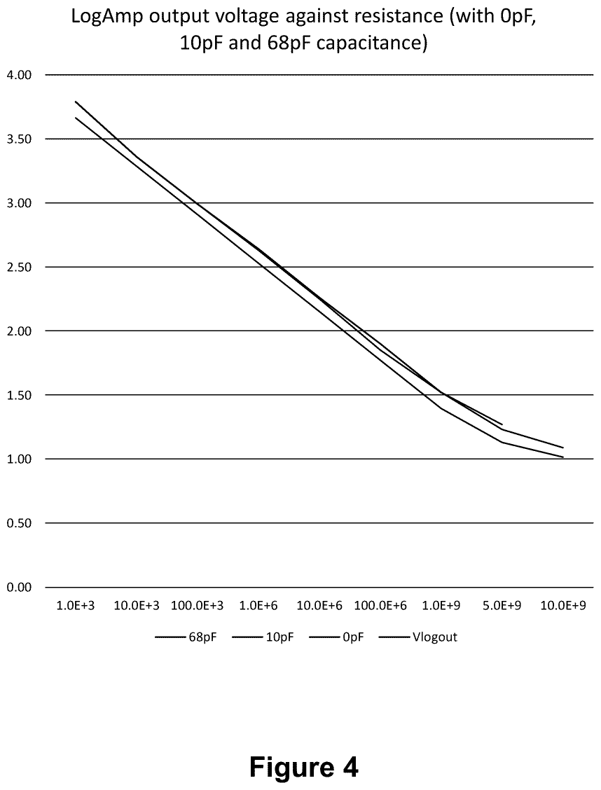

FIG. 4 is a graphical plot showing an example of the relationship between the resistance of a sensing element 120 and output from a resistance detector 150 in a sensor such as shown in FIG. 1. In particular, FIG. 4 presents results obtained from a simulation of using a resistance detector 160 incorporating a LOG114 logarithmic amplifier 120A to measure resistance. The X-axis is used to denote resistance values from 1 kOhm to 10 gOhm, i.e. spanning seven decades. The Y-axis is used to denote the voltage output from the logarithmic amplifier 120A having values in the range 1-4 volts. There are four lines plotted showing the variation of voltage output against known (simulated) resistance values. Three of these lines depict the measured voltage outputs from the log amplifier when three respective capacitances (0 pF, 10 pF and 68 pF) were attached across the resistance being measured. The Vlogout line represents the “ideal” log amplifier output based on the equation that is in the Data Sheet for the LOG114 device. The plot shows the 0 pF line (top) in red, the 68 pF line (middle) in light blue, and the Vlogout line (bottom) in dark blue. The legend makes reference to the 10 pF line which is in green, however this line is substantially identical to the red (top) line for 0 pF and so is hidden, i.e. not separately visible, in FIG. 4.

It can be seen that there is a linear relationship between the (log) resistance value and the voltage output. The voltage falls from approximately 3.75V to approximately 1.25V, namely by 2.5V, while the resistance rises from 10³ to 10¹⁰ Ohms, namely 7 decades. This is a voltage drop of ~0.36V per decade, which is comparable with the gradient of 0.375 from Equation 2 above. There is a small change in slope (gradient) for higher resistances, greater than 10⁹ Ohms. This may be an artefact of the increased susceptibility to noise at these levels of high resistance, as discussed above.

The lines for all three measured outputs for different values of capacitance are extremely close together with a variation in output voltage generally <<0.1V. This confirms that the resistance of the sensing element 120 can be accurately measured by the resistance detector 150 independently of the capacitance of the sensing element. The plot of the Vlogout line (bottom, dark blue) shows the measured results have a small offset from the “ideal” log amplifier voltage. This offset is typically of the order of 0.1V and can be considered as reflecting the constant c in Equation 1 (or Voffset in Equation 2). The offset may be allowed for through calibration of the sensor 50 and/or reduced by further refinement of the circuitry (including shielding, etc) for the resistance detector 150.

Overall, providing a logarithmic amplifier 110 in the resistance detector 150 supports a rapid, accurate and reliable measurement of sensing element resistance across multiple (e.g. 7-8) decades of resistance, as may be experienced in various sensing elements, independently of sensing element capacitance.

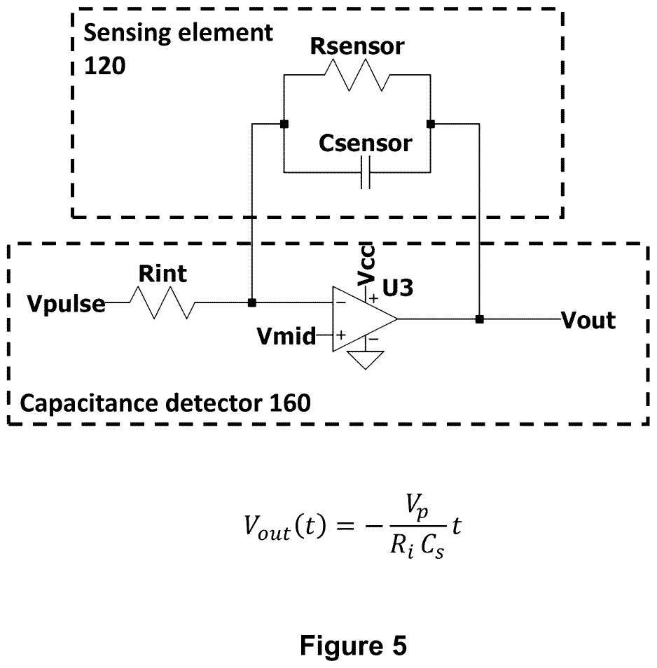

Turning now to the capacitance of sensor 160, FIG. 5 is a schematic diagram of an example of a capacitance detector 160 which may be used in the sensor of FIG. 1. The top portion of FIG. 5 shows the sensing element 120 which has an associated resistance and capacitance. As discussed above, the sensor 50 may include multiple sensing elements 120 which are to be read in turn. FIG. 5 further shows in schematic form an example implementation of the capacitance detector shown in FIG. 1.

The capacitance detector of FIG. 5 comprises an operational amplifier (op amp) (U3) which is configured as an integrator. In particular, the output voltage (Vₒᵤₜ) is an integral over time of the input voltage (Vᵢₙ). As shown in FIG. 5, the sensing element 120 in effect sits in the feedback loop of the op amp U3. The input to the capacitance detector 160 is provided as a voltage pulse, for example a square wave, top hat function, or similar. The capacitance detector 160 performs the capacitance measurement on the ‘on’ transition from low to high, in effect the step up of the input voltage pulse. There is subsequently a step down portion of the pulse, the ‘off’ transition from high back to low, however, this step down is not used for the capacitance measurement itself (other than to reset the input to allow another step up pulse to be utilised for the next measurement). The input voltage signal passes through a resister Rᵢₙₜ before reaching the op amp U3.

Intuitively, if Rₛₑₙₛₒᵣ is high (i.e. much larger than the range resistor (Rᵢₙₜ), then Rₛₑₙₛₒᵣ can be ignored. In this case, the feedback network from the sensing element 120 can be regarded as a pure capacitance (Cₛₑₙₛₒᵣ), which in effect converts the circuit of FIG. 5 into an integrator op amp circuit (see for example https://en.wikipedia.org/wiki/Op_amp_integrator and https://www.electronics-tutorials.ws/opamp/opamp_6.html). This integrator op amp provides the possibility to measure the sensor capacitance (Cₛₑₙₛₒᵣ) largely independently of the sensor resistance Rₛₑₙₛₒᵣ.

In operation, a voltage pulse (step) is supplied to the input voltage Vₒᵤₜ. Discounting the Rₛₑₙₛₒᵣ (as very large), when the voltage is first applied, the sensor capacitor has no charge and so acts akin to a short circuit. In this situation, the voltage at the inverting input to op amp U3 remains at zero and there is no current flow into this input (in accordance with ideal op amp behaviour). Therefore the (effective) current through the resistor, which is given by Vᵢₙ/Rᵢₙₜ, must equal the current through the capacitor, which is given by dQ/dt=−CdVₒᵤₜ/dt (assuming again ideal operation of the op amp, so that the output voltage corresponds to the voltage across the sensing element capacitance), hence Vᵢₙ/Rᵢₙₜ=−CdVₒᵤₜ/dt. Integrating both sides with respect to time gives

Vₒᵤₜ=−(1/Rᵢₙₜ Cₛₑₙₛₒᵣ)∫Vᵢₙdt,

in other words, the output voltage is the integral with time of the input voltage Vᵢₙ.

For the particular case of the input voltage comprising a single step at time T, such that Vᵢₙ goes from 0 before time T to a known, constant value (Vₚ), then Vₒᵤₜ has the form of a straight line ramp starting at time=T with a slope that it is dependent on the product Rᵢₙₜ Cₛₑₙₛₒᵣ (and the known size of the voltage step Vₚ for the input voltage pulse). In other words, there is a linear relationship between the ramp rise time and capacitance of the sensing element 120. Thus for known values of Vₚ and Rᵢₙₜ, the capacitance of the sensing element 120, namely Cₛₑₙₛₒᵣ, can be determined.

Significantly, this determination of the capacitance of the sensing element 120 does not depend on any time duration (such as measuring a time constant to determine RC). Rather, the value of RC (and hence Cₛₑₙₛₒᵣ) may be measured as soon as the ramp has increased enough to allow a determination of the slope (gradient) of the ramp. For example, assuming that we regard time T for the voltage pulse as representing time=0, then subsequently the output voltage Vₒᵤₜ may be measured at time t, and the slope is then given by Vₒᵤₜ/t (independent of any RC timing constant). In practice, measurements by the capacitance detector 160 of the capacitance of the sensing element 120 can be performed quickly with the op amp integrator, so that even for a sensing element having a high resistance, the measurement of capacitance typically takes no more than of the order of a few microseconds. Furthermore, there may be some ability to configure the timing to be used for the measurements by appropriate selection of Rᵢₙₜ and the step input magnitude, whereby the timing can be brought into a reasonable range for making the desired measurement of capacitance.

In some implementations, two comparators having set voltage levels (thresholds), one high, one low, are used to perform the determination of capacitance. In particular, when the output voltage passes the voltage level of the low level comparator, this may start a timer. Then, when the output voltage passes the voltage level of the high level comparator, this may stop the time. The capacitance of the sensing element 120 may be determined based on the (known) voltage difference between the low and high levels, plus the measured time for the voltage output to rise from the low level to the high level (plus knowledge of R and C). In an example implementation, the comparators may be implemented using TLV3601 devices available from Texas Instruments (TI)—see https://www.ti.com/product/TLV3601 and the associated data sheet. However, it will be appreciated that any other suitable comparators may be used instead. With these comparators, a change of capacitance at the 1 pFarad level may detected (when the resistance of the sensing element is low).

In this approach, the output voltage must pass the low and high thresholds in order to trigger the comparators. This can be arranged (inter alia) by increasing the pulse amplitude and/or by reducing the integration resistor. Both of these methods produce greater (steeper) ramp slopes. It will be understood that these steeper ramp slopes may provide more reliable activation of the comparators but typically they also require faster timing. Another approach is to have an additional comparator at mid-rail for timing the 50% rise time of an RC curve when the op amp is not reaching saturation. Having this mid-rail may impose quite stringent timing requirements to measure the full range of likely time values of practical interest (the time values may be as short as around 300 ps at one end of the range to achieve the desired resolution for measured capacitance values).

In some implementations, a time-to-digital converter (TDC) is used to make very accurate measurements of the time between start and stop pulses, such as produced from the two (low and high) comparators as discussed above. By way of example, the TDC7201 device available from Texas Instruments (TI) is able to perform such high accuracy time measurements (see https://www.ti.com/product/TDC7201 and the associated data sheet). This TDC has a timing resolution of 55 picoseconds with an example measurement range of 0.25 nanoseconds to 8 milliseconds (this is variable according to the particular mode of operation selected). Note that the TDC is primarily intended for range-finding applications, however, the ability to discern very short time intervals is also useful and relevant to the timing measurements for discerning the capacitance of a sensing element as described herein.

It has been found that the present approach is relatively insensitive to the parameters of the particular type or configuration of op amp (there are many different types of op amp available). For example, it is not necessary that the op amp has a high input impedance. In one example implementation, an OPA357 op amp from Texas Instruments (TI) has been used for the capacitance detector 160—see https: //www.ti.com/product/OPA357 and the associated data sheet. However, any other suitable op amp may be used instead according to the particular circumstances of any given implementation.

In the approach discussed above, the capacitance of the sensing element(s) is obtained independently of the resistance of the sensing element(s)—in effect by assuming that the sensor resistance is effectively infinite. However, as the resistance of the sensing element 120 decreases to become significant in comparison to the integration resistance, i.e. when Rₛₑₙₛₒᵣ is no longer >>Rᵢₙₜ, the behaviour of the capacitance detector 160 starts to deviate from the behaviour of an ideal integrator.

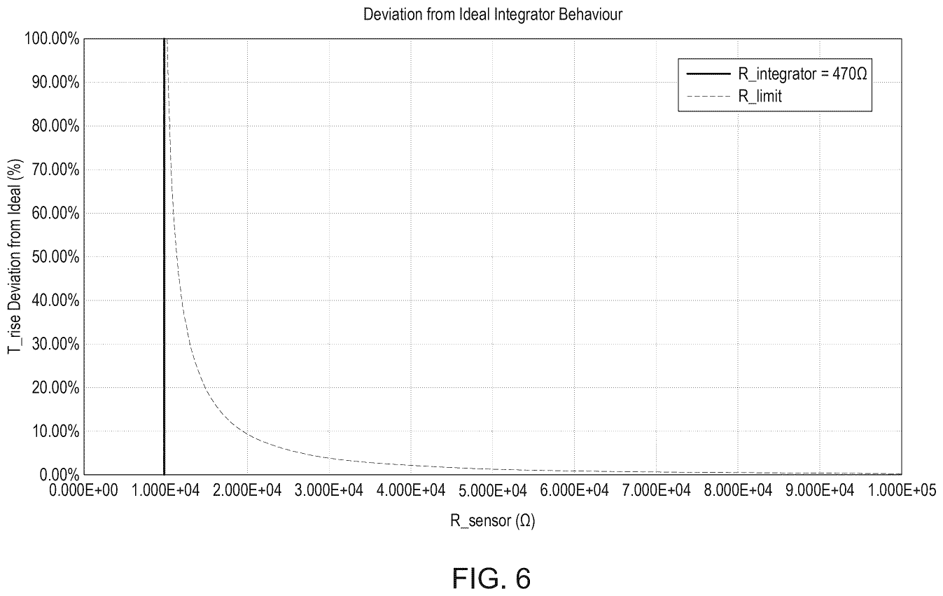

FIG. 6 is a graphical plot based on simulation results showing an example of this deviation of the op amp integrator from the behaviour of an ideal integrator due to the presence of resistance from the sensing element 120 as shown in FIG. 5. The X-axis represents the value in Ohms of the resistance of the sensing element 120, while the Y-axis shows the deviation in percentage terms of the actual rise time for a given resistance compared with the ideal rise time for a sensing element with very large resistance, i.e. with Rₛₑₙₛₒᵣ>>Rᵢₙₜ. (The rise time represents the time interval between triggering the first and second comparators as discussed above, which then directly impacts the measured value of capacitance for the sensing element 120). The plot of FIG. 6 is based on a value of Rᵢₙₜ of 470 Ohms in the capacitance detector, see FIG. 5. It can be seen that the percentage deviation (offset) is very small (at most a couple of per cent) down to a resistance of 40 kOhm. The deviation then increases to 10% for a resistance of 20 kOhm, after which the deviation rises increasingly rapidly so that 10 kOhm in effect represents a practical measurement limit with this configuration.

It should be noted that the plot of FIG. 6 only shows resistance up to 100 kOhm, however, as described above, the range of interest for resistance measurements may extend to around 25 GOhm. This illustrates that for a large portion of the desired resistance measurement range, say 25 GOhm down to 40 kOhm, the assumption of an ideal op amp integrator with the sensing element resistance in effect infinite works well and provides a high level of accuracy for measuring the capacitance of the sensing element. Furthermore, although FIG. 6 shows a practical limit for measuring capacitance of the sensor resistance being at least 10 kOhm, this can be reduced if so desired by lowering the value of Rᵢₙₜ, since the circuit operation depends on the ratio of Rₛₑₙₛₒᵣ to Rᵢₙₜ.

It is also noted that as shown in FIG. 6, the deviation in the measurement of rise time (and hence capacitance of the sensing element) can be characterised for any given values of Rₛₑₙₛₒᵣ and Rᵢₙₜ such as by simulating the behaviour of the op amp integrator circuit shown in FIG. 5. Furthermore, FIG. 6 shows a mapping of the calculated deviation in rise time for a known value of Rᵢₙₜ and an assumed value of Rₛₑₙₛₒᵣ. This mapping may be inverted to take a measured value of Rₛₑₙₛₒᵣ (obtained with the logarithmic amplifier as discussed above), and to use this to determine the deviation from the ideal rise time for this measured resistance value, for example, based on the plot of FIG. 6. Given this known deviation, it is possible to work backwards from the measured (actual) rise time to determine the corresponding ideal rise time, and hence obtain a reliable value for the capacitance of the sensing element (for example within a few per cent, such as within a range of 3-6%). Furthermore, although the curve of FIG. 6 is obtained from simulation, it may additionally (or alternatively) be derived from measurements of the actual rise times using capacitors of known capacitance to provide (further) calibration of the curve of FIG. 6.

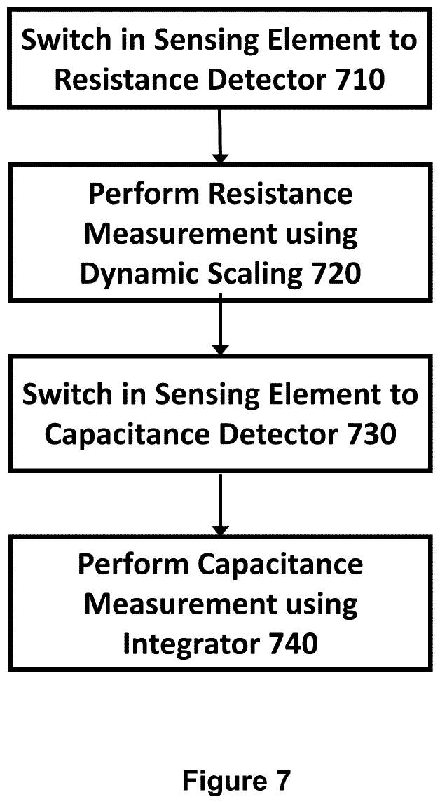

FIG. 7 is a flowchart showing an example of a method for operating a sensor 50 such as shown in FIG. 1 (or any other suitable sensor). The method commences with switching the sensing element 120 of the sensor into a resistance detector at operation 710. This switching can be done in any suitable manner using switching circuitry within the sensor 50 (typically under the control of the sensor control system 130). The resistance detector 150 is now used to make a measurement of the resistance of the sensing element 120 using logarithmic scaling as discussed above at operation 720. When this resistance measurement has been completed, the sensing element is switched out of the resistance detector 150 and into the capacitance detector 160 at operation 730. The capacitance detector 160 is now used to make a measurement of the capacitance of the sensing element 120 using an integrator as discussed above at operation 740. In some implementations, the capacitance detector 160 may incorporate a time-to-digital convertor (TDC) as discussed above.

When the capacitance measurement has been completed, the sensor is ready to return to the start of the process shown in FIG. 7, to perform updated resistance and capacitance measurements on the same sensing element, and/or to perform resistance and capacitance measurements on one or more additional sensing elements within. In the latter case, it will be appreciated that the switching circuitry is able to switch between the different sensing elements (as well as between the resistance and capacitance detectors as shown in FIG. 1).

In the above approach, the resistance measurement and the capacitance measurement are performed independently of one another. (The measured resistance may be used to adjust the measured capacitance at low resistance levels for the sensing element as described above, however, this adjustment does not impact the device measurement process per se, rather it is an adjustment applied to the outcome of this process). The resistance measurement and the capacitance measurement are generally performed in rapid succession of one another within a period time period, e.g. within a few (say 10 or 5) milliseconds of one another, sometimes within a few (say 10 or 5) microseconds of one another. For most sensing operations, this allows the resistance measurement and the capacitance measurement to be considered as contemporaneous of one another. Although FIG. 7 shows the resistance measurement as being performed prior to the capacitance measurement, in some implementations this ordering may be reversed.

An example implementation of a sensor 50 as disclosed herein may include battery & power management to produce voltage rails; a log amplifier associated with a reference voltage to measure the resistance of a sensing element, typically to an accuracy of around 5%, plus in some implementations an analog to digital converter (ADC) to acquire the output of the log amplifier; an op amp integrator for measuring capacitance plus in some implementations a time-to-digital convertor (TDC) for tight timing resolution; switching circuitry, for example using instrumentation multiplexes or reed relays, for swapping between the resistance detector 150 and capacitance detector 160 and/or for swapping between different sensing elements; two channels for providing a calibration resistance and a calibration capacitance; a processor for controlling operations such as converting ADC voltage to resistance and TDC results to capacitance, as well as controlling the sensor and providing additional functionality (including the sensor control system of FIG. 1); Bluetooth support; and temperature and humidity indicators. It will be appreciated that other sensors may utilise a different set of components, so the above set of components should not be regarded as limiting.

In addition, it is noted that the LOG114 device and other devices specifically identified herein are provided way of example only. In particular, it will be appreciated that other implementations may use other hardware components which differ from those specified herein but which provide similar functionality. (Such other hardware components may be supplied by the same vendor or by a different vendor from those hardware components specifically identified herein).

A sensor as disclosed herein may support a very wide range of resistance measurements, for example from 4500 to 25 GΩ (providing there is careful shielding at the higher levels of resistance). The capacitance measurements are typically performed with a range of 1 pF to 100 pF. The capacitance detector may include a TDC chip to provide fine timing resolution and detection down to the level of nanoseconds. A ±1 pF resolution may be achieved with a TDC chip measuring the circuit timings. In general, the timing (including setting times) for resistance measurements may be less than 200 μs for a resistance of ≤1 GΩ<200 μs, and less than 3 ms for a resistance of >1 GΩ<3 ms, while the timing for capacitance measurements is typically a few μs and may in some implementations be less than 1 μs.

These timings support fast switching between the resistance and capacitance detectors, as well as between different sensing elements. In particular, this allows effectively contemporaneous measurements (worst case within a few (say 10 or 5) ms of each other, in many cases within 200us of each other) of resistance and capacitance for a given sensing element. In addition, the sensor is able to cycle through obtaining corresponding R and C measurements for each sensing element in turn, with the duration of the cycle being sufficiently low to provide a high sampling frequency (and hence fine time resolution) for the output from each sensing element.

An example of a sensor 50 as described herein is an electronics device that can precisely and (near) simultaneously measure, with good temporal resolution, the capacitance and resistance of high impedance sensing elements which are located in an array of sensing elements and have resistances which range from gigaOhms down to Ohms and picofarad-level capacitances that vary over time. The resistance and capacitance are measured independently, but within ~10ms of one another. To achieve this, low impedance switching circuitry may be used to switch in specific circuits optimised for measuring resistance and capacitance separately, such as the resistance detector 150 and the capacitance detector 160 as described above.

The resistance may be measured using a logarithmic amplifier with a wide dynamic input range and on-chip temperature drift compensation. This technique relies on the current through the sensor (rather than the RC curve), thereby allowing a very quick measurement to be taken. The logarithmic amplifier converts the input current to an output voltage directly corresponding to the sensor resistance, which may then be measured using an analogue to digital converter and processed to provide the measured resistance.

The capacitance may be measured with pF resolution by switching the sensor into the feedback loop of an op-amp integrator configuration. When a step input is applied, the output of the integrator is a ramp with a slope dependent on the sensor capacitance, the step amplitude and the integration resistor, the latter two of which are fixed by design. Provided the sensor resistance is high in comparison to the integration resistor, the measured ramp rise time is proportional to the capacitance.

The integration resistor and step magnitude are selected in order to extend close to ideal integrator behaviour of the circuit to as low a value for the sensor resistance as possible (down to a few kΩ). The resulting rise times are of the order of 0.1 ns per pF change in capacitance. To time the rise times with sufficient resolution, a Time to Digital Converter (TDC) may be employed, which can time (measure) pulses as short as 0.25 ns with 55 ps resolution.

Accordingly, resistance and capacitance detectors such as described herein may be used with sensing elements that change resistance by orders of magnitude when exposed to an analyte. The sensing elements also have a simultaneous change in capacitance over time which is also to be measured (but typically with a lower scale of change compared to resistance).

Two further fixed resistance/capacitance channels may be provided to allow the processor to compensate for any minor circuit tolerance differences. This provides a form of calibration to help achieve consistent results from system to system. In some implementations, the measured resistance and capacitance values may be analysed using a machine learning (ML) system and having more consistent resistance and capacitance values helps the ML system to learn more quickly and to provide more consistent outputs.

Accordingly, a sensor 50 may be used to sense an environmental condition, which may involve sensing multiple physical parameters. The sensor 50 may comprise an array of sensing elements, all incorporated (for example) in a battery-powered, hand-held device. In one example, the environmental condition relates to health of soil, and the multiple physical parameters reflect the concentrations of different gases (volatiles). The sensor 50 may have multiple sensing elements which may include multiple copies of the same sensing element (for redundancy, better accuracy, etc) and additionally (or alternatively) different versions of the sensing element. For example, in the latter case, different sensing elements may be provided with different coatings which interact in different ways and/or amounts with the target analytes. The different sensing elements may be used, for example, to discriminate between different target analytes and/or to extend the set of target analytes which can be detected and measured by the sensor.

The sensor may be used for a gas sensor or an electronic nose, for example comprising a polymer semiconductor-based sensor array for the sensing elements. Such devices have a wide range of potential applications. Further, the sensor 50 may be used in the medical field for bio-sensors that are (for example) submerged in liquids to check for the presence or absence of target analytes.

In some implementations, an electronic nose may contain sensing elements specifically for measuring different substances, for example, each sensing element might be configured to detect the presence or concentration of a given gas. In other cases, the various sensing elements may not have such specific, individual measurement objectives, but rather readings from multiple sensors may be used in combination to provide a useful characterisation of a given analyte (perhaps based on an ML analysis). For example, in the case of a device to monitor soil health, the device may provide a yes/no output as to whether or not the soil is considered to be healthy. In other cases, the device may output a score (metric) which provides some numerical representation of overall soil health—e.g. the score might range from 1 to 10, with 1 representing poor soil, and 10 representing very healthy soil. Other implementations of such a device may provide output on multiple metrics relating to an environmental condition, where each parameter may be determined from one or more sensing elements. For example, such a sensor for the analysis of soil health might provide information on soil acidity/alkalinity, nutrient levels, water retention and/or microbe population(s).

Although the above description relates primarily to the context of determining soil health, it will be appreciated that sensors such as disclosed herein may be used in many other (different) contexts, such as for checking food products, for detecting contamination in various environments, for monitoring exhaust products, and so on. Different sensors may be used in different contexts and various aspects of the sensors may alter according to context. For example, the present application has cited a range of resistance and a range of capacitance to be measured for the sensing elements, but it will be appreciated that in other contexts and/or with other types of sensor, the range of resistance and/or capacitance to be measured may be adjusted accordingly.

In conclusion, while various implementations and examples have been described herein, they are provided by way of illustration, and many potential modifications will be apparent to the skilled person having regard to the specifics of any given implementation. Accordingly, the scope of the present case should be determined from the appended claims and their equivalents.

1. A sensor for measuring an environmental condition, comprising
a sensing element having a resistance and a capacitance which are sensitive to the environmental condition;
a first measurement circuit for determining the resistance of the sensing element using logarithmic scaling;
a second measurement circuit including an integrator amplifier for determining the capacitance of the sensing element; and
a switching circuit for switching the sensing element in turn into the first and second measurement circuits such that the resistance and capacitance can be determined within a predetermined time period.

2. The sensor of claim 1, wherein the first measurement circuit comprises a log amplifier.

3. The sensor of claim 1, wherein the first measurement circuit is configured to measure resistance over at least six decades.

4. The sensor of claim 1, wherein the first measurement circuit is configured to measure resistance over a range having:
a lower limit of no greater than 2500 ohms, and
an upper limit of no less than 1 gOhm.

5. The sensor of claim 1, wherein the integrator amplifier comprises an op amp integrator.

6. The sensor of claim 1, wherein the sensor is configured to provide a step change voltage to the integrator amplifier, and wherein the slope of the output from the integrator amplifier is dependent on the capacitance to be measured.

7. The sensor of claim 6, wherein the second measurement circuit further comprises a time to digital converter (TDC), and optionally wherein the TDC may be used to measure the slope of the output from the integrator amplifier according to claim 6.

8. The sensor of claim 1, wherein the sensor is configured to apply a correction to the measured capacitance based on the measured resistance.

9. The sensor of claim 8, wherein the correction is only applied if the measured resistance is below a threshold value, optionally wherein the threshold value is below 100 kOhms.

10. The sensor of claim 1, wherein the second measurement circuit is configured to measure capacitance over a range having:
a lower limit of no greater than 5 F, and
an upper limit of no less than 100 F.

11. The sensor of claim 1, wherein the operation of the first measurement circuit for determining resistance is performed independently from, and sequentially with, the operation of the second measurement circuit for determining capacitance.

12. The sensor of claim 11, wherein the resistance and capacitance can be determined within a predetermined time period of 0.1 s. 1 ms.

13. The sensor of claim 1. further comprising at least one channel for providing a known resistor for measurement by the first measurement circuit and/or for providing a known capacitor for measurement by the second measurement circuit as a mode of calibration.

14. The sensor of claim 1, wherein the sensor comprises multiple sensing elements, optionally wherein the number of sensing elements is in a range from 1 up to 6, and optionally within a range from 1 up to 20, optionally with a range from 1 up to 50.

15. The sensor of claim 14, wherein the sensor is configured to switch each sensing element in turn into the first and second measurement circuits.

16. The sensor of claim 15, wherein the sensing elements are repeatedly switched in turn into the first and second measurement circuits, and the time duration for all the sensing elements to be switched in turn into the first and second measurement circuits for measuring the resistance and capacitance of all the sensing elements is no greater than 1 second.

17. The sensor of claim 1, wherein the sensing element comprises an electronic nose comprising a polymer semiconductor-based sensor array.

18. The sensor of claim 1, wherein the environmental condition to be measured corresponds to soil health.

19. A method of using a sensor to measure an environmental condition, the sensor comprising a sensing element having a resistance and a capacitance which are sensitive to the environmental condition, the method comprising:
switching the sensing element into a first measurement circuit;
determining with the first measurement circuit the resistance of the sensing element using logarithmic scaling;
switching the sensing element into a second measurement circuit; and
determining with the second measurement circuit the capacitance of the sensing element using an integrator amplifier.

20. The method of claim 19, wherein the sensor comprises the sensor of claim 1.
